(19) United States
(12) Patent Application Publication
(10) Pub. No.: US 2002/0173683 A1
(43) Pub. Date: Nov. 21, 2002

(54) Asymmetric hydrogenation catalysts and processes

(76) Inventor: Peter Chen, Zurich (CH)

Correspondence Address: GREENLEE WINNER AND SULLIVAN P C, 5370 MANHATTAN CIRCLE, SUITE 201, BOULDER, CO, 80303, US

(21) Appl. No.: 10/050,613

(22) Filed: Jan. 16, 2002

Related U.S. Application Data
(60) Provisional application No. 60/262,550, filed on Jan. 16, 2001.
(60) Provisional application No. 60/306,276, filed on Jul. 17, 2001.

(51) Int. Cl. C07C 5/02; B01J 31/00; B01J 37/00

(52) U.S. Cl. 585/250; 585/275; 502/103; 502/117; 502/104

(57) ABSTRACT

Improved hydrogenation catalysts, catalyst formulations and methods employing them. Activity of Ru(II) complexes as hydrogenation catalysts is enhanced by addition of Lewis base as a cocatalyst.

### CROSS-REFERENCE TO RELATED APPLICATIONS

This application takes priority from U.S. provisional patent application serial No. 60/262,550, filed Jan. 1, 2001, and from U.S. provisional patent application serial No. 60/306,276, filed Jul. 17, 2001, both of which are incorporated by reference in their entirety herein.

### BACKGROUND OF THE INVENTION

Asymmetric catalysis has been reviewed in a recent monograph by Noyori (Asymmetric Catalysis in Organic Synthesis Wiley, N.Y., 1994). Although the field has generated a large amount of academic activity, industrial processes in which asymmetric catalysis performs sufficiently well to be cost-competitive with conventional resolution are still few, with L-DOPA and (-)-menthol being perhaps the most prominent early examples (J. Halpern Prec. Metals 1995, 411). In particular, asymmetric hydrogenation has attracted interest because of the potentially easy access to enantiopure secondary alcohols and amines via reduction of prochiral ketones and imines.

The most promising types of catalysts for asymmetric hydrogenation are Ru(II) complexes with at least one amine ligand, a class of compounds usually associated with the name of Noyori. Recent reviews by Noyori describe thoroughly the progress in the field (R. Noyori, S. Hashiguchi, Acc. Chem. Res. 1997, 30.97; R. Noyori, T. Okhuma, Pure Appl. Chem. 1999,71,1493; R. Noyori, T. Okhuma, Angew. Chem. Int. Ed. 2001 40, 40). There are two classes of Ru(II) complexes, exemplified by structures 1 (Class 1) and 2 (Class 2), each showing certain advantages as well as disadvantages. These classes are distinguished functionally as hydrogenation catalysts, at least in part, in the hydride sources that can be employed.

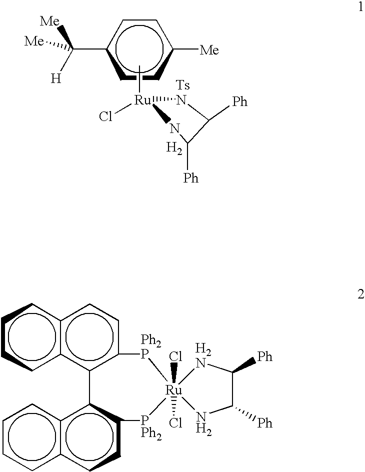

Complexes related to 1 have been reviewed (R. Noyori, S. Hashiguchi, Acc. Chem. Res. 1997, 30, 97). Several groups other than Noyori's have produced catalysts based on similar structural motifs, all functioning as transfer hydrogenation catalysts with similar performance, reducing ketones in isopropanol with excellent enantioselectivity upon activation with one or a few equivalents (relative to catalyst) of base, usually KOH or an alkali metal alkoxide. See: T. Ohta, S. Nakahara, Y. Shigemura, K. Hattori, I. Furukawa, Chem. Lett. 1998, 491; Y. Jiang, Q. Jiang, X. Zhang, J Am. Chem. Soc. 1998,120,3817; J. X. Gao, P. P. Xu, X. D. Yi, C. B. Yang, H. Zhang, S. H. Cheng, H. L. Wan, K. R. Tsai, T. Ikariya, J Mol Catal. A 1999,147,105; E. Mizushima, H. Ohi, M. Yamaguchi, T. Yamagishi, J. Mol. Catal. A 1999,149,43; D. G. I. Petra, P. C. J. Kamer, P. W. N .M. van Leewen, K. Goubitz, A. M. Van Loon, J. G. de Vries, H. E. Schoemaker, Eur. J Inorg. Chem. 1999,2335; D. Alonso, S. J. M. Nordin, P. Roth, T. Tamai, P. G. Andersson, M. Thommen, U. Pittelkow, J Org. Chem. 2000,65,3316; J. X. Gao, H. Zhang, X. D. Yi, P. P. Xu, C. L. Tang, H. L. Wan, K. R. Tsai, T. Ikariya, Chirality 2000,12,383.

While the utility of these class 1 catalsyts has been demonstrated in laboratory-scale reductions, and their activity, as measured by the substrate-to-catalyst ratio (S/C) typically ranges from 100 to 10,000, they are nevertheless less suitable for industrial applications because even having a S/C=10,000 places the catalyst only just at the borderline of cost-effectiveness (H. -U. Blaser, M. Studer, Appl. Catalysis A 1999,189,191), despite the ease by which the ligands and catalysts are prepared. Moreover, although a wide range of structural variation has been tried, these catalysts (Class 1 catalysts) do not, accept H₂ as a hydride source.

Mechanistic work on catalyst 1 has resulted in the isolation and structural characterization of intermediates 3 and 4 (K. -J. Haack, S. Hashiguchi, A. Fujii, T. Ikariya, R. Noyori, Angew. Chem. Int. Ed. Engl. 1997,36,285).

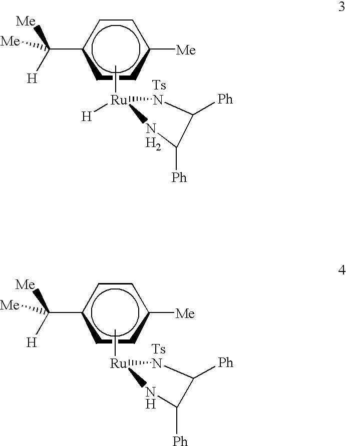

The mechanism, depicted below in Scheme 1 for the asymmetric reduction of acetophenone, is derived from a combination of spectrometric(J. A. Kenny, K. Versluis, A. J. R. Heck, T. Walsgrove, M. Wills, J Chem. Soc. Chem. Comm. 2000,99), structural, kinetic, and quantum chemical studies D. A. Alonso, P. Brandt, S. J. M. Nordin, P. G. Andersson, J Am. Chem. Soc. 1999,121,9580; M. Yamakawa, H. Ito, R. Noyori, J Am. Chem. Soc. 2000,122, 1466). In the context of the latter, all known experimental details have been conveniently summarized (M. Yamakawa, H. Ito, R. Noyori, J Am. Chem. Soc., 2000,122,1466). In the mechanism illustrated in Scheme 1, bidentate ligands are shown schematically. Of particular importance is the observation that 3 and 4 catalyze the transfer hydrogenation of ketones in the absence of base with the same activity and selectivity as the combination of catalyst 1 with base. The transition states 5a and 5b, and all of the species involved in the catalytic cycle, are mononuclear ruthenium complexes, with Noyori explicitly stating that the base plays no role beyond generation of the active species 3 and 4. It is important to note that an excess of base destroys the catalyst.

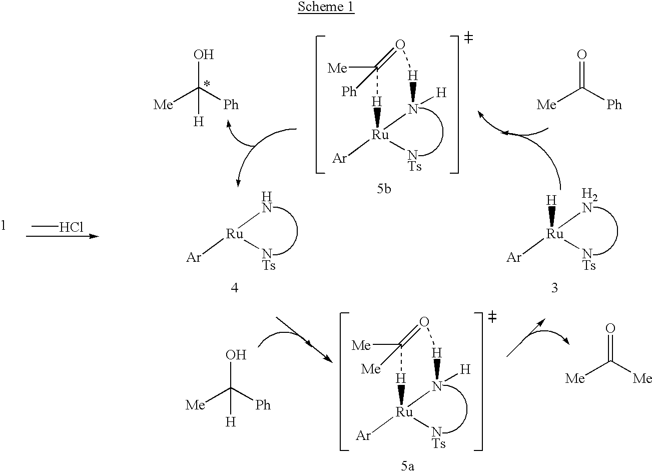

Complexes related to 2 have also been reviewed (R. Noyori, T. Okhuma, Pure Appl. Chem. 1999,71,1493)and described in certain patent applications (R. Noyori, K. Mikami, T. Ohkuma, Japanese Patent JP 241119/97, Sep. 5, 1997; European Patent EP 0 901 997 A1, Sep. 2, 1998). In contrast to Class 1 catalysts, these superficially similar Ru(II) complexes of Class 2 reduce ketones and imines with H₂ as the hydride source rather than isopropanol. The enantio- and chemoselectivity is comparable to those for Class 1, but the substrate-to-catalyst ratio (S/C) is much higher, with a record S/C=2,400,000 having been achieved (H. Doucet, T. Ohkuma, K. Murata, T. Yokozawa, M. Kozawa, E. Katayama, A. F. England, T. Ikariya, R. Noyori, Angew. Chem. Int. Ed. Engl. 1998,37,1703). One disadvantage of the Class 2 catalysts is the necessity for phosphine ligands. A more important limitation is that this class of catalysts requires a large excess of base (up to >1000 equivalents.) While the hydrogenation can proceed with stoichiometric (relative to catalyst) base, the high S/C ratio is only observed when 100 equivalents or more of base are used. Use of such a large excess of base poses a problem for base-sensitive substrates, limiting the scope of the catalytic reaction.

The mechanism for reduction of ketonic substrates is assumed to proceed by a transition state analogous to 5a or 5b. Importantly, there is no extant hypothesis that explains the divergent behavior of these two classes of Ru(II) complexes. Recently, Morris (K. Abdur-Rashid, A. J. Lough, R. H. Morris, Organometallics 2000,19,2655) has isolated and structurally characterized two putative intermediates related to 1.

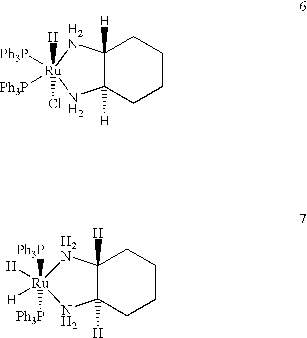

Both 6 and 7 reduce ketones in the presence of H₂ and because of this they are in Class 2. As in complex 2, complex 6 requires 10 or more equivalents of base for catalytic function; in contrast, 7 functions without any added base at all. For these two catalyst, however, S/C=5000 is reported, which is far below the performance of 2 under comparable conditions for the same substrates. Morris proposes a catalytic cycle similar to that involving 3 and 4, with the exception that H₂ coordination and heterolytic cleavage regenerates 7 after the step in which the ketone is hydrogenated.

Thus, no catalytic system for asymmetric hydrogenation using metal complexes has been reported which shows simultaneously the three properties: (1) high activity as defined as turnover comparable to Noyori Class 2 catalysts (2) the use of low levels of alkoxide base or, equivalently, a substantially weaker base than alkoxide or hydroxide, and (3) the use of H₂ as hydride source. All descriptions of the mechanism of such catalysis involve only mononuclear Ru(II) species in the catalytic cycle. The present invention provides improved methods and formulations for asymmetric hydrogenation which exhibit all three of the desired properties just mentioned.

### SUMMARY OF THE INVENTION

An improvement of asymmetric hydrogenation catalysts and processes is reported in which even unactivated prochiral carbonyl compounds and imines can be reduced to alcohols and amines enantioselectively. The invention is based on the discovery that addition of a Lewis acid cocatalyst, such as an electrophilic metal or metal cation, to a Ru(II) hydrogenation catalyst formulation (e.g., Ru(II) complex activated with base in excess over the base present results in a significant enhancement in catalyst turnover.

It is believed that Lewis acid addition results in generation or enhancement of the concentration of a bi- or trimetallic complex comprising a Ru(II) center, a second metal atom or cation, optionally a third metal atom or cation, and one or more bridging ligands coordinated between the Ru(II) center and the second and/or third metal atom or cation, and supporting ligands for the Ru(II) and the second and optional third metal atom or cation. Key improvements of the catalyst and its use in an asymmetric hydrogenation process over existing catalysts and processes are:

high catalyst activity, which renders the catalyst of the present invention more commercially viable with regard to cost;

reduction of the amount of alkoxide base used in the reaction, which is important when the substrate to be reduced contains base-sensitive functional groups or structures resulting in expanded scope of utility for the catalyst; and

acceptance of H₂ as a hydride source which reduces cost and is generally more practical for commercial application of hydrogenation catalysts

The improved catalysts of this invention, and processes using them, are components in the manufacture of enantiomerically-enriched or enantiopure pharmaceuticals and fine chemicals, estimated to comprise a world market with approximately US$ 90 billion annual sales.

The improved catalysts and catalyst formulations of this invention can be provided by combination of Ru(II) complexes with at least one equivalent with respect to the Ru(II) complex of base and with a Lewis acid in excess over the base or bases present. The Lewis acid can, for example, be an oxophilic Lewis acid which binds well to oxygen, one or more electrophilic metal cations and/or a Lewis acid containing boron, aluminum or tin. The base can be, but is not limited to, a hydroxide, an alkoxide or a hydride base or mixtures thereof. The base can also be a non-ionic base such as DBU.

This invention provides improved catalysts and catalyst formulations useful generally for hydrogenation, preferably hydrogenation employing H₂ as the hydride source, and which are particularly useful for catalysis of asymmetric hydrogenation to generate products, particularly alcohols and amines, enantioselectively. Preferably enantioselective products are generated with enantiometric excess. of 30% or more, 40% or more or 50% or more. More specifically, the invention provides a method for reducing or hydrogenating a carbonyl bond and catalysts and catalyst formulations for carrying out the method. The method of the invention can, for example, be used for the asymmetric hydrogenation of a prochiral carbonyl bond, such as for the reduction of a ketone.

In specific embodiments the invention provides catalysts and catalytic hydrogenation methods employing the catalysts wherein a hydrogen-cleaving Ru(II) catalyst is prepared by combining a Ru(II) complex of formula:

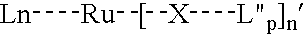

wherein:

X is a heteroatom, including but not limited to nitrogen, oxygen, L″ is an optionally substituted hydrocarbyl or a ligand containing one or more oxygen, nitrogen, phosphorous, boron, aluminum, or tin atoms, p is an integer dependent upon the valency of X and L″; L, independent of other L in the complex, is a ligand selected from a halogen, a solvent molecule, a ligand containing one or more oxygen, nitrogen or phosphorous chelating atoms, a ligand containing one or more boron, aluminum or tin atoms, an arene, particularly a π-bonded arene which may be optionally substituted, and dihydrogen; and n and n′ are integers with n′ ranging from 1 to 6 and n ranging from 0 to 5 dependent upon the valency of L ligands and the value of n′;

with at least one equivalent with respect to the Ru complex of a base and a Lewis acid present in excess over the base. The base can be present in an amount ranging from at least 1 equivalent to about 100 equivalents with respect to the Ru complex. More preferably the base is present in an amount ranging from at least 10 equivalents to about 100 equivalents with respect to the Ru complex. The base can be present at all ranges intermediate between at least about 1 and 100equivalents, e.g., ranging from about 10-50 equivalents, or 20-40 equivalents with respect to he Ru(II) complex. In general the amount of base is selected to achieve desired catalyst activity while minimizing detrimental effects on the Ru(II) complex, its ligands, or the substrate and products of the catalytic reaction.

Ru(II) complexes useful in the catalytic methods and formulations of this invention include those of the above general formula which comprise (1)one or more phosphine ligands; (2)one or more amine ligands; (3) one or more ether ligands; (4) an oxazoline ligand; (5) one or more diamine ligands (6) a π-bonded arene ligand or combinations thereof. One or more of L or L″ can be chiral.

In a specific embodiment, one or more of L, L″ or both, or portions of L, L′ or both form a cation binding site for an electrophilic metal cation (e.g., an alkali metal cation or alkaline earth metal cation. For example, aryl or substituted aryl groups on L and L″ can together form a site for binding of a metal cation, e.g., by π-bonding to the aryl rings.

Catalysts and catalyst formulations prepared by combination of Ru(II) complexes with base and Lewis acid are employed for hydrogenation by combination with a hydride source, typically in an appropriate solvent, such as an alcohol, and combination with a compound to be hydrogenated. The temperature of the hydrogenation, the duration of the reaction and other reaction conditions can be readily optimized. The catalysts, catalyst formulations and methods of this invention preferably employ hydrogen as the hydride source.

The invention also provides hydrogenation catalysts that contain boron, provided in boron-containing ligands as illustrated herein below. Of particular interest are Ru(II)-based catalysts having boron-containing ligands in combination with phosphine ligands. Catalysts provided include those having mono- and bidentate boron-containing ligands in combination with multidentate phosphine ligands. These complexes and catalysts and catalyst formulations prepared by combining these complexes with base and/or Lewis acid including electrophilic metals and metal cations, can be employed in catalytic hydrogenation reactions.

Further, the invention provides catalysts, catalyst intermediates, catalytic formulations and catalytic methods using them in which one or more of the ligands of the Ru(II)-complex are replaced with dihydrogen. Dihydrogen Ru(II) complexes are believed to be formed in situ on combination of the components of the catalyst formulations of this invention with a hydride source.

The methods of this invention can employ Class 2 Ru(II) complexes as defined and illustrated herein and as described and illustrated in review articles cited herein in combination with a base and/or a Lewis acid to enhance catalyst activity.

The methods of this invention can employ Class 1 Ru(II) complexes as defined and illustrated herein and as described and illustrated in review articles cited herein in combination with a base and/or a Lewis acid to enhance catalyst activity and/or to facilitate acceptance of hydrogen as a hydride source in catalytic hydrogenation employing the complexes.

The invention further provides hydrogenation catalysts and catalytic methods using them that contain no phosphine ligands, but which can employ H₂ as the hydride source. Ru(II)-based hydrogenation catalysts without phosphorus-containing ligands have not been previously reported. In particular, the invention provides Ru(II)-based catalysts having an oxazoline ligand the formula:

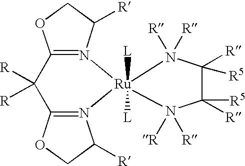

wherein L, independent of the other L, is a ligand which can be selected from halogens or solvent molecules; R, independent of other R in the complex, can be hydrogen, or an optionally substituted hydrocarbyl group, particularly an optionally substituted alkyl group or an optionally substituted aryl group, and more particularly a small alkyl group (straight-chain, branched or cyclic) having 1 to 6 carbon atoms (e.g., methyl groups, ethyl groups, propyl groups, etc.); R′, independent of other R″ in the complex, can be an optionally substituted hydrocarbyl group, particularly an alkyl group or an aryl group, either of which can be optionally substituted, preferred R″ are aryl groups and optionally substituted aryl groups; R″, independent of other R″ in the complex, can be a hydrogen or an optionally substituted hydrocarbyl group, preferred R″ are hydrogen and small alkyl groups having 1-6 carbon atoms (e.g., methyl group, ethyl group, propyl group, etc.); R⁵, independent of other R⁵ in the complex, can be optionally substituted hydrocarbyl groups including alkyl groups and aryl groups and optionally substituted alkyl and aryl groups, preferred R⁵ are aryl or optionally substituted aryl groups.

Any two or more of R, R′, R″ or R⁵ can be linked to form a ring. Aryl ligands of these complexes may contain one or more aromatic rings which can include heteroaromatic and/or fused rings, any of which may be optionally substituted particularly with one or more halogens, one or more alkyl groups, one or more alkoxy groups or one or more halogenated alkyl groups.

Also provided are catalysts and catalyst formulations formed by combination of the Ru(II)-oxazoline complexes above with base and/or Lewis acids and those complexes, including dihydrogen complexes, formed by combination of the Ru(II)-oxazoline complexes above with base and/or Lewis acids in the presence of a hydride source.

Catalysts of the complexes described herein and catalysts and catalyst formulations prepared by combining these complexes with bases and/or electrophilic metals or metal cations or more generally with Lewis acids, particularly those containing boron, aluminum or tin, can be employed for catalytic hydrogenation of carbonyl-containing substrates (e.g., ketones and imines).

### DETAILED DESCRIPTION OF THE INVENTION

The mechanistic picture for asymmetric hydrogenation with Ru(II) catalysts that has been described in the art is, at best, incomplete. New aspects of the mechanism as reported herein provide the basis for significant improvements in asymmetric hydrogenation catalysts and processes using them.

When catalyst 2 is activated by the nonionic base DBU (1,8-diazabicyclo[5.4.0]undec-7-ene) instead of the normal alkoxide bases, a second electrophilic metal cation is a necessary and sufficient cofactor for hydrogenation activity, both in initiation and subsequent turnover of the catalytic cycle. In other words, the metal cation associated with the base plays a role in the catalytic reaction beyond that of a mere spectator, and moreover, plays a role in turnover which is entirely ignored in the prior art. Given the previously known labilization of H₂ coordinated to a Ru(II) center towards hetereolytic cleavage(M. S. Chinn, D. M. Heinekey, J Am. Chem. Soc. 1987,109,5865; E. P. Capellani, P. A. Maltby, R. H. Morris, C. T. Schweitzer, M. R. Steele, Inorg. Chem. 1989,28,4437; D. M. Heinekey, W. J. Oldham Jr., Chem. Rev. 1993,93,913), the metal cation effect on the activity of 2 may be reasonably attributed to a further increase in the propensity for heterolytic cleavage of H₂ in the close proximity to a strong electrophile through polarization or inductive effects (DBU is a substantially weaker base than an alkoxide (pKa=11.8 for the conjugate acid in water). The addition of alkali metal salts to the solution of 2 and DBU leads to metalation of the amido site in activated 2.)

Moreover, this new information indicates, that the inability of the Noyori catalysts similar to 1 to accept H₂ as the hydride source likely derives from insufficient acidification of coordinated H₂. As is evident from the ability of 7 to catalyze hydrogenation of ketones without added base (and without a second metal cation), the choice of an appropriate ligand set can lead to sufficient acidification of H₂ to achieve turnover. However, as is evident from the inferior S/C value, the turnover occurs under marginal conditions unsuited to commercial operation.

Based on the new mechanistic information, improved catalysts with the structural subunit 8a are provided:

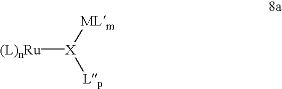

in which Ru is a ruthenium atom in the +2 oxidation state, M is an electrophilic metal atom or cation including, but not limited to, the alkali or alkaline earth metal cations, a transition metal (in addition to the Ru shown), boron, aluminum, or tin; X L″ₚ is a heteroatom bridging ligand where X is a heteroatom (i.e., non-carbon atom); L , independent of other L, is selected from a halogen, a solvent molecule, a dihydrogen, an arene, particularly a π-bonded arene, a ligand containing one or more oxygen, nitrogen, and/or phosphorus chelating atoms, such as alkyl or aryl phosphorous ligands, alkyl or aryl amines, ether or cyclic ether groups, one or more ligands containing one or more oxygen, one or more nitrogen and/or one or more phosphorous atoms in combinations, an additional X-L″ₚ ligand; L″, independent of other L″, are substituent groups compatible with the heteroatom X, including but not limited to, optionally substituted hydrocarbyl groups which can include, but are not limited to, alkyl groups, which may be straight-chain, branched or cyclic, alkene groups which may be straight-chain, branched or cyclic and which may contain more than one double bond wherein the double bonds may be conjugated and aryl groups which may be single-ring, multiple-ring or fused-ring systems, or combinations thereof, all of which alkyl and aryl groups are optionally substituted, for example with halogens, alkyl groups, alkoxy groups, halogenated alkyl groups or in which one or more CH₂ groups or CH groups can be substituted among others with O, N, B, Al, or Sn atoms or C═O, NH moieties; and L′ is a ligand which may be solvent molecules, halogens, optionally substituted alkoxides (—OAlkyl) or optionally substituted aryloxides (—OAr), anionic ligands containing halogen, oxygen, or nitrogen or ligands containing one or more oxygen, one or more nitrogen, and/or one or more phosphorous atom, such as optionally substituted alkyl or aryl phosphorous ligands, optionally substituted alkyl or aryl amines, optionally substituted ether or cyclic ether groups, one or more ligands containing one or more oxygen, one or more nitrogen and/or one or more phosphorous atoms in combinations, any of which may be chiral. Multiple L″, L′ and/or L in formula 8a can be the same or different. Two or more L, L′ or L″ can be linked to form a ring.

The integers n, m and p are the number of ligands L, L′ and L″, respectively. These integers range generally from 0 to 6, but depend, as will be appreciated by those in the art, upon the valences of Ru, M and X, the number of bridging ligands present and the number and valency (i.e., number of chelating atoms in the ligand) of any bi- or multidentate ligands that may be present. For example, a π-bonded arene group has a valency of 3.

The function of one or more L, L′ or XL″ₚ can be provided by another of L, L′ or XL″ₚ which is a bi- or multidentate ligand, e.g., a bridging ligand X-L″ₚ (e.g., OR or N(R)₂, where R can be various optionally substituted alkyl or aryl groups) can be provided by a portion of a multidentate L or L′. Each L, L″, and/or XL″ₚ can, for example, be a monodentate ligand with one atom which binds to the metal (Ru or M), bidentate ligand having two chelating atoms which bind to Ru, M or both (functioning as a bridging ligand when binding between Ru and M), a tridentate ligand having three chelating atoms that bind to Ru, X or both, a quatradentate ligand having four chelating atoms that bind to Ru, M or both .

The ligands Lₙ, L′ₘ, and L″ₚ may optionally be linked together to form chelating ligands that bind to one or both of Ru and/or M and optionally also provide the bridging ligand XL″ₚ, e.g., bi- or multidentate ligands. Complexes of the above formulas may have two or more bridging ligands XL″ₚ (the number of bridging ligands being dependent upon the selection of M) in these cases one or more L, L′, XL″ₚ ligands or portions of one or more of such ligands can function as the additional bridging ligands.

In an alternative embodiment, improved trimetallic catalysts with the structural subunit 8b are provided:

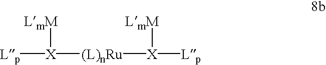

wherein M represents a second and a third metal atom or cation (in addition to Ru(II)), wherein the metal atom or cations M are as defined above in formula 8a and M's in formula 8b may be the same or different. The definitions of the other variables in formula 8b are as defined above in formula 8a and multiple L″, L′ and L in the formula may be the same or different.

M is metal or metal cation that is preferably electrophilic. The term electrophilic is intended to refer to metals that will not displace Ru(II) from the complex, but which will substantially enter the complex as described rather than remain solvated. Preferred electrophilic metal cations are alkali and alkaline earth cations, particularly K⁺and Na^{+.}

Further provided is a catalytic formulation in which two or more components are combined. The first component is a complex containing the structural subunit 9.

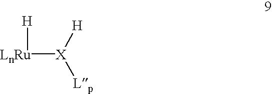

The definitions of L, L″, X, n and p are the same as those given for formulas 8a and b. This component may be added to the formulation or generated in the formulation by combination of two or more sub-components. The second component is one or more equivalents (preferably at least one equivalent or more) with respect to 9, of a compound that is a Lewis acid. For example, the second component can be either (1) one or more salts in which the cation is an electrophilic metal cation, preferably an alkali or alkaline earth cation, and the anion is a weakly or noncoordinating anion, or (2) one or more neutral Lewis-acidic compounds, such as a boronate ester, an aluminate or a tin compounds, such as, esters, e.g., Sn(OR)₄ or halides, e.g., SnCl₄. The second component may be one or more equivalents (preferably at least one equivalent or more) of a salt of an electrophilic metal cation or a mixture of salts of two or more metal cations. The second component may be a neutral Lewis-acidic compound or a mixture of two or more neutral Lewis-acidic compounds. The Lewis acid may be an oxophilic Lewis acid with an affinity for binding to oxygen. The second component may also be one or more equivalents (preferably at least one equivalent or more) of a mixture of (1) and (2). The formulation may be provided in an appropriate solvent, or a mixture of solvents or may contain an appropriate solvent or mixture of solvents. The formulation may also be provided with additional components to enhance reactivity or reaction yield.

Further provided is a catalytic formulation in which three or more components are combined. The first component is a complex containing the structural subunit 10.

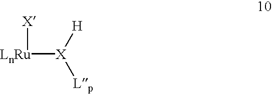

The definitions of L, X, L″, n and p are the same as those given for 8a and b. X′ is halogen, alkoxide, or another good leaving group comparable to a halogen or alkoxide group, as is understood in the art. The first component may be added to the formulation or it may be generated in the formulation by addition or two or more sub-components. The second component is one or more equivalents, with respect to the Ru complex, of a base or mixture of bases, preferably, but not limited to, a hydroxide, alkoxide, or a hydride base (See, Gordon, A; Ford, R. The Chemist's Companion: A Handbook of Practical Data, Techniques, and References, (1972) Wiley pages 67-71 and references therein for general information about bases and about the relative strength of bases). The third component is one or more equivalents (preferably at least one equivalent or more), with respect to 10, of a compound that is a Lewis acid. For example, the third component can be either (1) one or more salts in which the cation is an electrophilic metal cation, preferably an alkali or alkaline earth cation, and the anion is a weakly coordinating or noncoordinating anion, or (2) one or more neutral Lewis-acidic compound such as a boronate ester, an aluminate or a tin compound, such as esters, e.g., Sn(OR)₄ or halides, e.g., SnCl₄. The third component may be one or more equivalents of a salt of an electrophilic metal cation or a mixture of salts of two or more metal cations. The third component may be a neutral Lewis-acidic compound or a mixture of two or more neutral Lewis-acidic compounds. The third component may be mixture of (1) and (2). The formulation may be provided in an appropriate solvent, or a mixture of solvents or may contain an appropriate solvent or mixture of solvents. The formulation may also be provided with additional components to enhance reactivity or reaction yield.

The term “Lewis acid” or Lewis acidic compound” as used herein is intended to encompass the meaning of this term as currently appreciated in the art and includes any substance that will accept an electron pair from an electron pair donor. Molecules with one or more vacant orbitals on a central atom, such as compounds of boron, aluminum, beryllium zinc and tin, are common Lewis acids. The term Lewis acid includes species that are neutral and positively charged and includes oxophilic Lewis acids which may be neutral or charged and which bind oxygen well. Oxophilic Lewis acids include among others, early transition metal cations, in the first two transition series, the lanthanides, the alkali metal and alkaline earth metal cations, as well as some neutral Lewis acids based on boron or aluminum.

The term base is used generically herein to refer to chemical species that are proton acceptors and includes charged and neutral species. Bases of various strengths, as defined, for example, by pKa values of the conjugate acids of the base, can be employed. Addition of one base to a solvent can generate another base, e.g., addition of a base to an alcohol solvent can generate the alkoxide base. When the term base is used herein in reference to the compositions of formulations or combinations of components it refers to base added to a formulation and to any other base that may be generated on preparation of the formulation or combination of components.

Further provided complexes useful as catalysts and useful in catalyst formulations of the specific structures, 11-16, prepared either in situ or as isolated compounds.

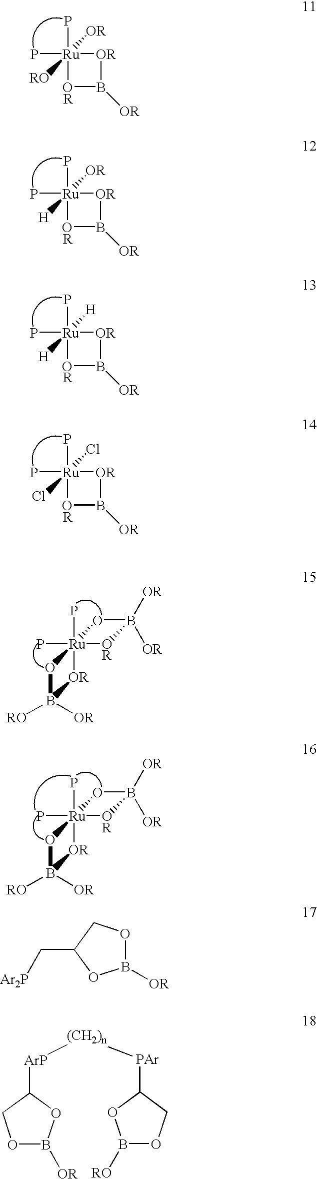

where the phosphine ligands may be monodentate or bidentate, as schematically indicated. The phosphine ligands in structures 11-16 are understood to carry sufficient other functional groups, e.g. aryl or other hydrocarbyl groups, to satisfy valence. The groups R are hydrocarbyl, including alkyl (straight-chain, branched or cyclic), alkenyl (including dienes) and aryl groups (one or more aromatic rings, which can include fused rings) which are optionally substituted with various groups including halogens, alkyl groups, halogenated alkyl groups, aryl groups and halogenated aryl groups and may optionally be further connected to the phosphines present to produce improved binding. Lines linking P and O, or two Ps above are hydrocarbyl groups particularly alkyl, cycloalkyl, alkenyl and cycloalkenyl groups, which may be substituted with one or more halogens, alkoxy groups, alkyl groups halogenated alkyl groups, aryl groups or halogenated aryl groups. Additional exemplary substituents for nitrogen-containing, phosphorous-containing and/or oxygen-containing ligands are illustrated in the formulas of Scheme 2

Specifically provided for are ruthenium complexes of ligands 17 and 18 and products thereof formed upon treatment with bases including, but not limited to, alkali metal hydroxides and alkoxides. Ruthenium complexes of ligand 17 include those with one or two of ligands 17 optionally in combination with L. L′ or X-L″p as noted above. Also specifically provided are ruthenium complexes of ligands 33-41 in Scheme 2 and products thereof formed upon treatment with base and/or Lewis acids cocatalysts, including, but not limited to, treatment with alkali metal hydoxides and alkoxides.

Catalytic processes using 8a or 8b, or the above-cited formulations containing 9 or 10, or those containing Ru(II) complexes of this invention in which a carbonyl-containing compound is reduced are provided. In specific embodiments, H₂ is employed in the reaction as a hydride source. More specifically processes using 8a or 8b, or the above-cited formulations containing 9 or 10, in which an aldehyde or a ketone or an imine are reduced to alcohols or an amine by H₂ are provided. Moreover processes using 8a or 8b, or the above-cited formulations containing 9 or 10, in which a prochiral ketone or prochiral imine is reduced to an enantiomerically-enriched chiral alcohol or chiral amine, respectively, by H₂ are provided. Additionally, analogous processes using any of the catalysts of formulas 11-16 and Ru complexes of the ligands of formulas 17 and 18, above are also provided.

One or more of L in structures herein can be dihydrogen. Dihydrogen complexes are believed to be formed on combination of Ru(II) complexes with base and/or Lewis acid in the presence of hydrogen.

In specific embodiments, Lₙ in the formulas herein can include monodentate and bidentate nitrogen or phosphorous ligands, particularly organonitrogen and organophosphorous ligands, including but not limited to, alkyl and aryl amines, alkyl and aryl phosphines, diamines and bisphosphines. Mono- and bidentate nitrogen and phosphorous ligands can be optionally substituted with halogens, alkyl groups, alkoxy groups, aryl groups, aryloxy groups, among others.

Lₙ can represent, for example, a combination of amine, phosphine and halogen ligands. In particular, Lₙ, can represent a combination of a bidentate phosphine, a bidentate amine and halogen. Lₙ can represent a combination of a diamine ligand, monodentate phosphines and halogen. Lₙ can represent a combination of a diamine ligand, bidentate phosphine and halogen. Amine ligands include alkyl amines, aryl amines, aryl-substituted alkyl amines and alkyl-substituted aryl amines. Specific amine ligands include optionally substituted ethylenediamine ligands, particularly those which are substituted with phenyl, which in turn may be substituted or other aromatic substituents. Alkyl amines can include those with optionally substituted straight-chain, branched, or cyclic aliphatic groups or alkene groups. Aryl amines include those with one or two nitrogens in an aromatic ring and may include fused ring systems or a combination of optionally substituted aromatic and alicyclic rings.

In further specific embodiments, Lₙ can include mono- and bidentate oxygen-containing ligands, nitrogen-containing ligands or ligands containing both O and N including, but not limited to, oxazolines. Specifically provided for are complexes of formula 19A and products thereof formed upon treatment of 19A with a combination of bases and salts in alcoholic solvent as asymmetric hydrogenation catalysts and catalyst formulations.

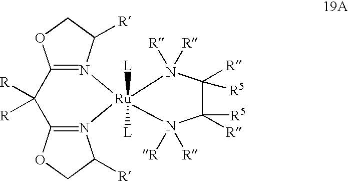

wherein L, independent of the other L, is a supporting ligand which can be selected from halogens or solvent molecules; R, independent of other R in the complex, can be hydrogen, or an optionally substituted hydrocarbyl group, including, but not limited to, an alkyl group or an aryl group, particularly a small alkyl group having 1 to 6 carbon atoms (e.g., methyl groups, ethyl groups, propyl groups, etc.) which are straight-chain branched or alicyclic; R′, independent of other R′ in the complex, can be an optionally substituted hydrocarbyl group, particularly an alkyl group or an aryl group either of which can be optionally substituted, preferred R′ are aryl groups and optionally substituted aryl groups; R″, independent of other R″ in the complex, can be a hydrogen or an optionally substituted hydrocarbyl group; R″ can, for example, be optionally-substituted alkyl, alkenyl, aryl, or aryl-substituted alkyl groups. R″ may be alkyl groups having 1 to 6 carbon atoms which are straight-chain, branched or alicyclic. R″ may also be (or form a portion of ) a bicyclic alkyl group, preferred R″ are hydrogen and small alkyl groups having 1-6 carbon atoms (e.g.,methyl, ethyl, propyl, etc.); R⁵, independent of other R⁵ in the complex , can be an optionally substituted hydrocarbyl group, including but not limited to, alkyl groups, alkenyl groups, and aryl groups and optionally substituted alkyl, alkenyl and aryl groups, preferred R⁵ are aryl or optionally substituted aryl groups. Aryl ligands of 19A may contain one or more aromatic rings which can include heteroaromatic and/or fused rings, any of which may be optionally substituted, for example with, substituted with one or more halogens, one or more alkyl groups, one or more alkoxy groups or one or more halogenated alkyl groups. R⁵ and/or R″ groups may be linked together to form a ring. Amine ligands and/or the oxazoline ligands can be chiral, achiral or racemic.

In other specific embodiments, complexes of formula 19B and products thereof formed upon treatment of 19B with a combination of bases and salts in alcoholic solvent optionally in the presence of hydrogen are provided as asymmetric hydrogenation catalysts and catalyst formulations:

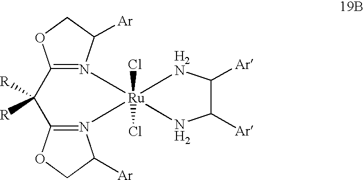

where Ar and Ar′, independently, are optionally substituted aryl groups which may contain one or more aromatic ring which can include heteroaromatic and/or fused rings, any of which may be substituted, for example, with one or more halogens, one or more alkyl groups, one or more alkoxy groups or one or more halogenated alkyl groups. Catalyst formulations of this invention include dihydrogen complexes of complexes 19A and 19B formed on combination of these complexes with base and/or Lewis acid in the presence of a hydride source, such as hydrogen.

Exemplary, phosphorous-containing, nitrogen-containing and oxygen-containing ligands are shown in formulas 20-32 below and in formulas 33-41 in Scheme 2. Catalysts of this invention include those of formulas 8a and 8b wherein any of Lₘ are ligands as shown in formulas 20-32 below and in formulas 33-41 in Scheme 2. Note that the multidentate ligands illustrated may provide for one or more L, L′ or XL″ moieties.

Phosphine ligands include alkyl phosphines, aryl phosphines, aryl-substituted alkyl phosphines and alkyl-substituted aryl phosphines.

Specific phosphine ligands include those of formulas:

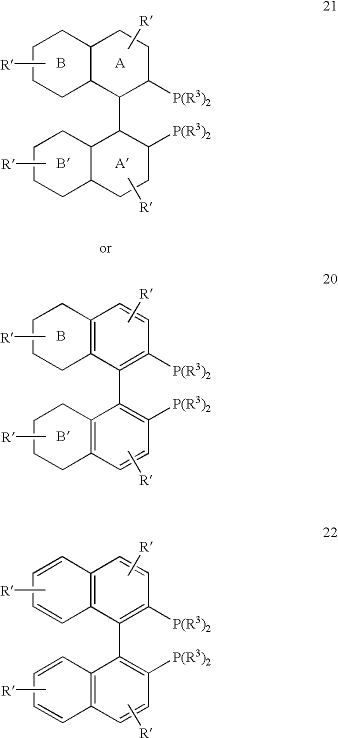

where A and A′, B and B′ can be optionally-substituted aromatic or alicyclic rings and R′ and R³ are optionally-substituted alkyl, aryl, alkyl-substituted aryl or aryl-substituted alkyl groups including lower alkyl groups (having 1-6 carbon atoms), phenyl rings and optionally-substituted phenyl rings. R′ can also be H. R′ can represent one or more different substituents on a given ring and R′ on the same or different rings may be the same or different. R³ groups on the same or different P may be the same or different groups and two or more R³ groups may be linked together to form a ring.

Phosphine ligands further include:

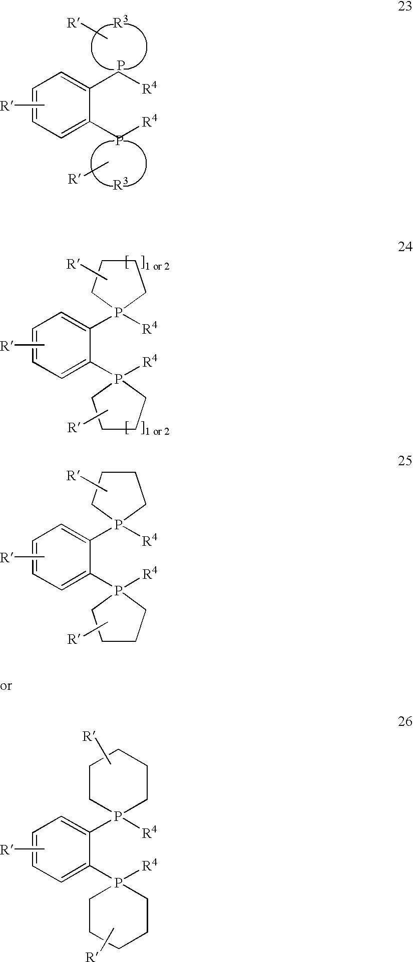

where R′ can represent one or more non-hydrogen substituents that are the same or different on one or more rings. R′ can be H or optionally-substituted alkyl, including alkyl having 1 to 6 carbon atoms, optionally-substituted alkoxy, including alkoxy having 1 to 6 carbon atoms, or optionally-substituted aryl or halogen.

Phosphine ligands can also include:

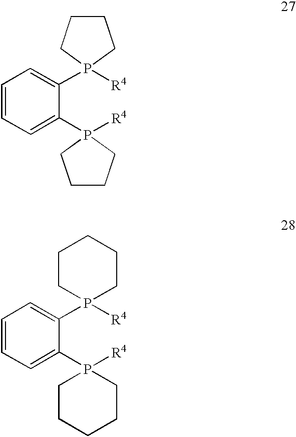

Phosphine ligands can be chiral, achiral or racemic. Other phosphine ligands include those of Scheme 1.

Amine ligands include those of structure:

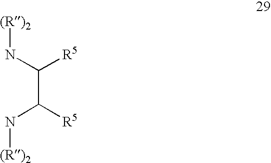

where R⁵ groups may be the same or different and include optionally-substituted alkyl, alkenyl or aryl groups. Two R⁵ may be linked together to form a ring. R″ at the same or different positions may be the same or different groups and can be hydrogens or optionally-substituted alkyl, alkenyl, aryl, or aryl-substituted alkyl groups. R″ may be alkyl groups having 1 to 6 carbon atoms which are straight-chain branched or alicyclic. R″ may be a bicyclic alkyl group. Amine ligands can be chiral, achiral or racemic.

More specifically amine groups include:

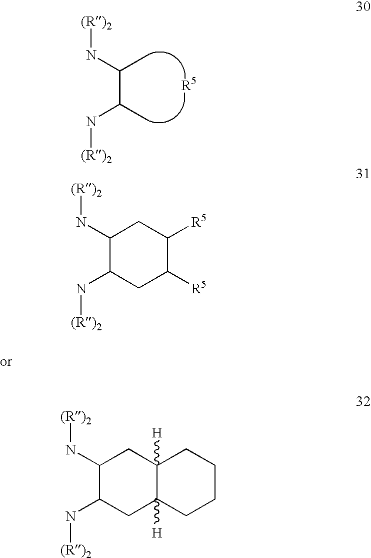

where R⁵ are alkyl, alkenyl or aryl rings and R″ is as defined above. In general, Lₙ may be chiral groups or include, chiral groups.

The term substitution as used in “optional substitution” generally includes substitution with any functional group or groups that does not substantially interfere with the catalytic reactivity or stability of the catalysts or catalyst formulations herein. Substitution includes among others, substitution with one or more halides, alkyl, phenyl, or alkoxy groups (each of which may in turn be fully or partially substituted with halide). Alkyl and alkoxy substituents can include straight-chain, branched or alicyclic groups. Phenyl substituents include optionally-substituted phenyl groups, such as 4-alkyl or 4-alkoxyphenyl or 3,5-dialkyl or 3,5-dialkoxphenyl groups. Optional substitution in hydrocarbyl groups, alkyl groups, alkene groups and alkylaryl groups includes substitution of one or more —CH—, CH₂—or —CH₃ groups in the group with O, N, S or other heteroatoms or C═O, CO₂, NH, or NH₂ groups.

The term hydrocarbyl as used herein and particularly as used in referring to R and L groups in formulas herein is used broadly to include optionally-substituted groups comprising saturated, unsaturated and aromatic hydrocarbons, including alkyl, alkenyl, alkynyl, and aryl (including aromatic) groups, as well as alkyl- or alkenyl-substituted aryl groups and aryl-substituted alkyl or alkenyl groups. One or more —CH—, CH₂—or —CH₃ groups in the hydrocarbyl group can be substituted with O, N, S or other heteroatoms or C═O, CO₂, NH, or NH₂ groups. Optional substituents include, among others, halide, alkyl, alkoxy, partially halogenated alkyl or aryl groups, and perhalogenated alkyl and aryl groups.

Catalytic processes of this invention include those using complex 8a or 8b, above and those using the formulations combining 9 or 10 with other components, as recited above, in which H₂ is the hydride source, and those which either (1) require the use of significantly less alkoxide base than expected (in view of the prior art) or the use of significantly weaker base than expected (based on the prior art) and which exhibit high S/C ratio. A high S/C ratio is greater than or equal to about 1,000. Preferably S/C is greater than 10,000, or greater than 100,000 and more preferably is greater than 500,000.

Formulations herein are prepared in solvent. An appropriate solvent is one that facilitates dissolution of substantially all components and does not interfere with desired reaction. Preferred solvents facilitate the desired hydrogenation reaction. More specifically alcohols, such as isopropanol, can be employed as the solvent. However, the reactions herein need not be done in a solvent that provides a hydride source. Other solvents, such as benzene, if they facilitate appropriate solvation of components in a given reaction can be employed.

In formulations of this invention, a Lewis acid is provided in excess over base and the base is provided at one or more equivalents with respect to the Ru(II) complex. Excess Lewis acid (more than one equivalent of base) can be a small excess (1 to about 5 equivalents) or a significant excess, greater than 10 equivalents. A large excess up to 1,000 equivalents may be used. More typically the amount of Lewis acid will range from about 5-100 excess over base. In general, the amount of excess Lewis acid is adjusted to optimize desired catalytic reaction without detrimentally affecting the substrate or product of the reaction.

More specifically a component of formulations herein is an excess of a salt of the cation M, particularly where M is an electrophilic metal cation (preferably Na⁺ or K⁺). The salt is present in excess over the base The excess may be small or significant or large. More typically, excess salt represents a 5- 100-fold excess over the base. The anion of the salt is preferably weakly coordinating or noncoordinating as understood in the art. Preferred anions are weakly basic or nonbasic as understood in the art.

In formulations of this invention, yet another component is one or more equivalents with respect to the Ru(II) complex of a base, particularly an alkoxide base A small excess typically is less than a 5-fold excess and more typically less than a 2-fold excess of base. A significant excess of base ranges between about 10-100 equivalents. In case of the use of a weaker base, a larger amount of the base may be beneficial to the reaction, as can be computed using known pKₐ values and standard equilibrium expressions.

Except as noted herein and consistent with the disclosure herein, the catalyst complexes and formulation of this invention are employed in hydrogenation reactions and asymmetric hydrogenation reaction as catalysts under conditions as described in the prior art.

The Ru-complexes in the catalysts and catalyst formulations of this invention can be assessed using mass spectrometric methods, for example, as illustrated in Hinderling et al. Angewandte Chem. (1999)111(15):2393-2396, which is incorporated by reference herein.

### EXAMPLES

### Example 1

### Asymmetric Hydrogenation with trans-RuCl₂[(S)-binap][(S,S)-dpen](2) in the presence of Lewis Acids

The results of solution-phase reactivity studies employing catalyst 2 are provided in Table 1. These studies were performed in thick-walled Pyrex pressure tubes fitted with a Bourdon-tube manometer on a stainless-steel head fitted with high-pressure valves (Whitey SS-43MA-S4, specified up to 200 bar). The solution, typically 2.9 ml, was degassed by three freeze-pump-thaw cycles, and then magnetically stirred in a temperature-controlled oil bath. The integrity of the apparatus was tested with H₂ up to 6 bar and found to be gas-tight with negligible pressure drop for periods up to 48 hours. Test reactions with 2 (3 mg), tBuOK (10 equivalents), and acetophenone (660 mg, 2000 equivalents) in isopropyl alcohol (2.9 mL, 2.0 M with respect to the ketone) for 4 h under H₂ (5 bar) at 50° C., , afforded (R)-1-phenethanol quantitatively with 90% ee, as measured by capillary gas chromatography with a chiral column (CE Instruments GC8000 TOP with a 30 m ×0.25 mm Supelco beta-DEX 120 column, the injector at 200° C., and the FID detector at 250° C.) A rather low S/C˜2000, corresponding to a millimolar concentration of catalyst, was chosen to adjust the reaction rate to a convenient range. After confirmation that the hydrogenation proceeded as expected, kinetic studies were performed by monitoring the rate of H₂ consumption over a period of several hours with or without various additives.

Although the conversion of the pressure drop per unit time to turnover frequency (TOF) requires a precise cell volume, the pressure drops themselves do serve as a relative rate measurement (By estimating the cell volume to be ˜20 ml, one gets an estimate of the TOF based on the pressure drop. For 1.3 bar pressure drop in 30 min, the TOF is approximately 700 hr⁻¹.) For each of the measurements, an initial rate, defined as the rate within the first 15 minutes after temperature equilibration and saturation with H₂ to 5 bar (t₀), is recorded. The second rate corresponds to the maximum rate of H₂ consumption (t₁).

The results in Table 1 must be considered with a particular equilibrium in mind. Using the pKa value for isopropanol (18, T. H. Lowry, K. H. Richardson, Mechanism and Theory in Organic Chemistry, 2^{nd} . ed., Harper & Row, New York, 1981, p. 266) and that of the conjugate acid of DBU (11.8, DBU=1,8-diazabicyclo[5.4.0]undec-7-ene, G. Häfelinger, F. K. H. Kuske, in The Chemistry of Amidines and Imidates, vol. 2, Chap. 1, S. Patai, Z. Rappoport, ed., Wiley, N.Y., 1991) the protonated fraction of DBU, and hence the concentration of isopropoxide, as a function of DBU concentration in neat isopropanol can be calculated. For a 1 mM concentration of 2, 100 equivalents of DBU would give approximately 1 mM isopropoxide (1 equivalent with respect to 2). Interestingly, a thousand-fold decrease in catalyst concentration to 1 μM, 100 equivalents of DBU would result in approximately 30 equivalents of isopropoxide relative to catalyst. Similarly, for a 1 mM and 1 μM solution of catalyst, 1000 equivalents of DBU would generate roughly 3 and 100 equivalents of isopropoxide, respectively. Both DBU and isopropoxide are present as bases when DBU is added to isopropanol. Thus, even in the (hypothetical) limiting case in which DBU only promotes dehydrochlorination, but does not participate further in the reaction, except through its contribution to isopropoxide concentration, at least one equivalent of isopropoxide is present.

As mentioned above, hydrogenation catalysts in the same family as 2 display high activity under mild conditions, outstanding enantio- and chemoselectivity, and a remarkable substrate-to-catalyst ratio (S/C). Noyori has proposed a novel mechanistic explanation for reversible hydride transfer between Rull amine complexes and secondary alcohols which rationalizes the activity and selectivity in the transfer hydrogenation catalysts, typically (arene)Ru(diamide) complexes, which differs sharply from the usual mechanism found in rhodium and iridium complexes (A. S. C. Chan, J. J. Pluth, J. Halpern, J Am. Chem. Soc. 1980, 102,5952; J. Halpern, J. Organomet. Chem. 1980,200,133; J. Halpern, Science 1982,217, 401; B. Bosnich, N. K. Roberts, Adv. Chem. Ser. 1982,196,337; J. M. Brown, P. A. Chaloner, D. Parker, Adv. Chem. Ser. 1982,196,355; H. B. Kagan, Asymmetric Synthesis 1985,5,1; K. E. Koenig, Asymmetric Synthesis 1985,5,71; J. M. Brown, Chem. Soc. Rev. 1993,25). Isolation of the presumed intermediates, as well as computational evidence, all support the proposed mechanism as at least plausible for transfer hydrogenation (J. A. Kenny, K. Versluis, A. J. R. Heck, T. Walsgrove, M. Wills, J Chem. Soc. Chem. Comm. 2000, 99;D. A. Alonso, P. Brandt, S. J. M. Nordin, P. G. Andersson, J. Am. Chem. Soc. 1999,121, 9580; M. Yamakawa, H. Ito, R. Noyori, J. Am. Chem. Soc. 2000,122,1466.)

The proposed mechanism does not however, explain why 2 and its close relatives can cleave dihydrogen, and moreover, why they display activity high enough to be catalysts with S/C ratio in excess of 1,000,000. A mechanistic scheme consistent with the experimental results in Table 1 is shown in Scheme 3 illustrated for the reduction of acetophenone in isopropanol with a potassium alkoxide as the inorganic base. The role of the potassium cation can be played by another suitable Lewis acid. In Scheme 3, both transfer hydrogenation as well as dihydrogen-cleaving routes are shown for the cases of the reaction with or without a Lewis acid cocatalyst. Because even the hydrogenation reactions catalyzed by 2 are performed in isopropanol, and the hydride transfer from the activated form of 2 to acetophenone is the microscopic reverse of a putative dehydrogenation of a secondary alcohol, one can already infer that 2 takes dihydrogen as a hydride source in preference to isopropanol because dihydrogen cleavage is accelerated relative to dehydrogenation of isopropanol (T. Okhuma, H. Ooka, S. Hashiguchi, T. Ikariya, R. Noyori, J. Am. Chem. Soc. 1995,117,2675.) In other words, for the catalytic cycle involving the earlier transfer catalysts-the upper half of Scheme 3-step (g) cannot compete with step (b), even if binding of H₂, step (f), is favorable. A process which accelerates dihydrogen cleavage until it dominates the competing isopropanol dehydrogenation necessarily leads to a catalyst, such as 2, which is more active, as measured by either turnover frequency or S/C, than the previous transfer catalysts.

The accelerated cleavage of dihydrogen corresponds in Scheme 3 to a dominance of steps (j) and (n) over step (p). While dihydrogen typically binds only weakly to neutral 16-electron RulI complexes, some cationic complexes bind H₂ quite strongly, e.g. [CpRu(dmpe)H₂]^{+ is stable in hot THF, with H}₂ displaced only slowly in refluxing acetonitrile. Moreover, dihydrogen coordinated to Rull can be strongly activated to heterolysis if the metal center is made sufficiently electron-deficient, i.e. if the complex is mono- or dicationic, but nevertheless capable of back-donation into the σ*H—H orbital of dihydrogen. The pKₐ of bound H₂ in [CpRu(dmpe)(H₂)]+ has been measured by Heinekeyto be˜5 (the pKₐ=17.6 in acetonitrile as given in M. S. Chinn, D. M. Heinekey, J. Am. Chem. Soc. 1987,109,5865 has been corrected to its value in water by subtraction of 12.5.) Similarly, Morris and coworkers (G. Jia, A. J. Lough, R. H. Morris, Organometallics 1992, 11,161 where pKₐ was determined by equilibrium measurements in THF and corrected to water) found pK_{a =}9.2 for [Cp*Ru(dppm)(H₂)]+. The variation of the pKₐ of H₂ bound to [CpRu(L)₂H₂]+for different phosphines L correlates systematically with electron-deficiency at the ruthenium center (E. P. Capellani, P. A. Maltby, R. H. Morris, C. T. Schweitzer, M. R. Steele, Inorg. Chem. 1989,28,4437.) Dicationic RU^{II} complexes (E. Rocchini, A. Mezzetti, H. Rüegger, U. Burkhardt, V. Gramlich, A. Del Zotto, P. Martinuzzi, P. Rigo, Inorg. Chem. 1997,36,711; T. A. Luther, D. M. Heinekey, Inorg. Chem. 1998,37,127.) show even more extreme behavior with respect to H₂. The same effect has been suggested to be operative in certain hydrogenases (R. T. Hembre, S. McQueen, J. Am. Chem. Soc. 1994,116,2141.) Given that the ruthenium amide complexes, Ru-N in Scheme 3, are neutral Ru(II) complexes, one would expect them to bind H₂ substantially more poorly than their cationic congeners. Moreover, the bound dihydrogen should be substantially less acidified than in [CpRu(dmpe)H₂]⁺. Step (g) in Scheme 3 is the deprotonation of bound dihydrogen by the amide ligand on ruthenium. There are no quantitative data availableon the basicity of the amide nitrogen, but one may make an estimate by two procedures. By considering the pKₐ of a primary ammonium cation—coordination of the amine to a Ru^{II} center is modeled in this estimate by protonation—one sees that the N—H bond of a primary amine is acidified from pKₐ from ˜30 to ˜10, a change of 20 orders of magnitude (E. M. Arnett, Prog. Phys. Org. Chem. 1963,1,223) which is consistent with the effect seen in other Ru and Os ammine complexes. The acidification of bound amines is also observed for Ru and Os ammine complexes in which the metal is in either the +2 or +3 oxidation state. G. Navon, D. Waysbort, J Chem. Soc. Chem. Comm. 1971,1410; J. D. Buhr, H. Taube, Inorg. Chem. 1980,19,2425; P. Bernhard, A. M. Sargeson, F. C. Anson, Inorg. Chem. 1988,27,2754; F. S. Nunes, H. Taube, Inorg. Chem. 1994,33,3116; P. Bernhard, D. J. Bull, H. B. Buirgi, P. Osvath, A. Raselli, A. M. Sargeson, Inorg. Chem. 1997,36,2804.

Alternatively, if one observes that activation of 2 requires 10 equivalents of potassium alkoxide when 2 is 1 mM, but 10,000 equivalents of base for 1 μM catalyst, then, with the assumption that full activation is represented by ˜90% deprotonation of the N—H moiety, one can compute pKₐ˜14 for a ruthenium amine complex. It should be noted that the requirement that the ruthenium amine remain deprotonated is presumably the reason for the enormous molar excess of base needed in the cases where S/C ratio is very high. The otherwise unexplained need for 10,000 equivalents of base when the catalyst is 1 μM is a consequence of the concentration-dependence of the proton-transfer equilibrium. The absence of significant dihydrogen cleavage in the absence of alkali metal cations indicates that the ruthenium amide, Ru—N, is insufficiently basic to heterolytically split dihydrogen efficiently enough to compete with the alternative isopropanol dehydrogenation reaction. In the absence of isopropanol, reduction of ketones with cleavage of dihydrogen can be observed in the absence of alkali metal cations because the transfer hydrogenation no longer competes. Morris' dihydride reduces ketones with dihydrogen in benzene. K. Abdur-Rashid, A. J. Lough, R. H. Morris, Organometallics 2000,19,2655.

On the other hand, coordination of the alkali metal cation to the ruthenium amide should withdraw electron density from the amide ligand, and hence ultimately from the ruthenium center, rendering coordinated dihydrogen more acidic. Moreover, the product of the previous cycle of reduction, a ruthenium amide with coordinated potassium alkoxide, places the basic alkoxide in an ideal position to deprotonate coordinated dihydrogen through a six-centered cyclic transition state, i.e. step (n).

Examination of either the x-ray crystal structure of 2, or a MMX-optimized computed structure finds that the N—H moiety that is close to syn-periplanar to the Ru—Cl (later Ru—H) bond has the amine proton situated between two face-to-face aryl rings, one from the diphenylethylenediamine and one from the phosphine, with the center-to-center distance between the two rings being ˜5.6 Å. Moreover, the midpoint between the rings lies ˜2 Å from the nitrogen. When the amine ligand is deprotonated, the amide nitrogen and the two aryl rings form a preorganized cation binding site for potassium, based on cation-arene interactions (J. C. Ma, D. A. Dougherty, Chem. Rev. 1997,97,1303; G. W. Gokel, S. L. De Wall, E. S. Meadows, Eur. J Org. Chem. 2000,2967).

Similar structures, albeit not catalytic, in which alkali metal cations are π-bound to aryl groups in Ru or Os complexes have been crystallographically characterized (G. P. Pez, R. A. Grey, J. Corsi, J Am. Chem. Soc. 1981,103,7528; J. C. Huffiman, M. A. Green, S. L. Kaiser, K. G. Caulton, J Am. Chem. Soc. 1985,107,5111, supporting the presence of such a binding site in complexes of this invention. The observed order for reaction acceleration, K>Na˜Rb>Li, would also be explained by the presence of metal cation-specific binding site in 2 by aryl groups from the two ligands on the ruthenium. Lewis acidity alone would have predicted Li>Na>K>Rb. Interestingly, the crown ether experiments for hydrogenation catalyzed by 2/tBuOK are less clearcut than the equivalent ones using 2/DBU/KBAF. A likely explanation is that crown ether sequestration of K+ in the former case not only removes the alkali metal cation, but also breaks up aggregation and ion-pairing (.Zavada, M. Pankova, Coll. Czech. Chem. Comm. 1978,43,1080.oftBuOK) with concomitant increase in the alkoxide basicity and partial compensation for the reduced H₂ consumption rate. The effect would be absent in the latter case.

TABLE 1
Relative rate of H2 consumption (bar h⁻¹)
in the catalytic hydrogenation of acetophenone with 2 for various conditions and additives*

| | Conditions | Rate at | | Addition at t₁ | | | Addtion at t₂ | |
|---|---|---|---|---|---|---|---|---|
| Entry | (eguiv) | t₀ | t₁(min) | (eguiv.) | Rate at t₁ | t₂(min) | (equiv.) | Rate at t₂ |
| 1 | 10 tBuOK | 2.8 | 30 | — | 3.6 | — | — | — |
| 2 | 2 tBuOK | 1.8 | 60 | 400 [18]crown-6 | 0.8 | 75 | 450 tBuOK | 1.0 (at t₂ = 135) |
| 3 | 100 DBU, 10 KBAF | 0.45 | 180 | 10 [18]crown-6 | 0.00 | 240 | 10 KBAF | 0.20 |
| 4 | 100 DBU, 10 NaBAF | 0.20 | 165 | 10 [18]crown-6 | <0.05 | 315 | 10 KBAF | >0.10 |
| 5 | 10 tBuOLi | 1.40 | 60 | — | 2.00 | | — | — |
| 6 | 10 tBuONa | 2.00 | 30 | — | 2.80 | | — | — |
| 7 | 10 tBuOK | 2.80 | 30 | — | 3.60 | | | |
| 8 | 10 tBuORb | 2.00 | 30 | — | 2.60 | | — | — |
| 9 | 100 DBU | 0.00 | 150 | — | 0.00 | | — | — |
| 10 | 100 DBU, 10 LiBAF | 0.00 | 120 | — | 0.00 | | — | — |
| 11 | 100 DBU, 10 NaBAF | 0.20 | 60 | — | 0.30 | | — | — |
| 12 | 100 DBU, 10 KBAF | 0.45 | 30 | — | 0.50 | | — | — |
| 13 | 100 DBU, 10 | 0.20 | 60 | — | 0.30 | | — | — |
| | RbBAF | | | | | | | |
| 14 | 100 DBU, 100 LiBAF | 0.23 | 60 | — | 0.16 | | — | — |
| 15 | 100 DBU, 100 | 3.00 | 40 | — | 3.63 | | — | — |
| | NaBAF | | | | | | | |
| 16 | 100 DBU, 100 KBAF | 3.33 | 30 | — | 6.00 | | — | — |
| 17 | 1000 DBU, 100 | 4.00 | 40 | — | 4.44 | | — | — |
| | NaBAF | | | | | | | |
| 18 | 1000 DBU, 100 | 4.29 | 30 | — | 7.20 | | — | — |
| | KBAF | | | | | | | |

*Each row describes an experiment which proceeds with the designated addition of a reagent or rate measurement at the marked time. Quantities of base and additives are expressed in mole equivalents relative to 2. All reactions were performed at 5 bar H₂ pressure at 50° C. BAF is the tetrakis(3,5-bis(trifluoromethyl)phenyl)borate anion.

Table 1 shows several clear results. While a base is needed for dehydrohalogenation of 2, alkoxide alone is insufficient for high activity. An alkali metal cation is also needed for high activity. The experiments adding crown ether show that the alkali metal cation is needed for turnover, not only for initiation. The different alkali cations influence the activity in the order K>Na˜Rb>Li. An increase in the alkali metal cation concentration with a constant amount of base results in higher activity. Moreover, the results show that addition of a Lewis acid, in the present example, alkali metal cations, in excess of the amount of alkoxide present increases hydrogenation activity. The results of Table 1 have been presented in R. Hartmann and P. Chen Angew. Chem. Int. Ed. 2001,40(19):3581, which is incorporated by reference in its entirety herein.

Noyori's catalyst 2 was synthesized from the dimeric complex [RuCl₂(C₆H₆)]₂ (M. A. Bennett, A. K. Smith, J Chem. Soc. Dalton Trans. 1974,233), (S,S)-1,2-diphenylethylenediamine [(S,S)-dpen,] and 1,1′-binaphthalene-2,2′-diylbis(diphenylphosphane) [(S)-binap], and purified by crystallization (H. Doucet, T. Okhuma, K. Murata, T. Yokozawa, M. Kozawa, E. Katayama, A. F. England, T. Ikariya, R. Noyori, Angew. Chem. Int. Ed. Engl. 1998,37,1703.) Spectroscopic data matched those reported in the literature. tBuOK and tBuONa were purchased from Fluka and used as received. tBuOLi, and tBuORb were prepared by reaction of either the hydride or the elemental metal with the appropriate alcohol. 1,8-Diazabicyclo[5.4.0]undec-7-ene (DBU) was purchased from Fluka and distilled under exclusion of air prior to use. [18]Crown-6 was dried by heating to 50° C. under high vacuum overnight. NaBAF (BAF=tetrakis(3,5-bis(trifluoromethyl)phenyl)borate) was synthesized by literature procedures (S. Bahr, P. Boudjouk, J Org. Chem. 1992,57,5545.). The remaining BAF salts were prepared from NaBAF by ion exchange as described by Nishida et al, Bull. Chem. Soc. Japan 1984,57, 2600.

Each row of Table 1 represents an experiment in which 1, dissolved in distilled and degassed solvent, was saturated with H₂ at 1 bar. Addition of the remaining initial reagents was followed by pressurization of the reaction cell to 5 bar with H₂ and vigorous stirring for 5 minutes. Control runs had shown that, in the absence of reaction, this time was sufficient for full saturation of the solution with H₂. At this point, the pressure vessel was sealed, and the pressure drop noted over a 10-15 minute period. The pressure in the vessel was kept at 5 bar subsequently by reopening the vessel to the H₂ source at 5 bar. Additions of the remaining reagents was done similarly. The maximum rate of H₂ consumption was found to occur at differing times, depending on the activity of the catalyst, due to the depletion of the substrate.

### Example 2:

### Asymmetric Hydrogenation Employing Ru-oxazoline-dpen Complexes General Synthesis of the Complexes

[RuCl₂(p-cymene)]₂(0.5 eq.), DPEN (1 eq.) and the bis-oxazoline ligand (1 eq) were weighed out in a drybox and dissolved in dry, degassed diglyme to form a solution with a concentration of approximately 0.09 M in ligand. The solution was refluxed for 15 minutes. Upon cooling to room temperature, crystals of the complex formed. The crystals were filtered off, washed with pentane, redissolved in chloroform, and then recrystallized by addition of pentane. The yield, depending on the particular bis-oxazoline ligand, was 30-50%. The complexes were subsequently characterized by ¹H nmr in CDCl₃ at −65° C. and by ESI-MS from CH₂Cl₂. Note: the Ru-oxazoline-DPEN complexes are sensitive to oxygen.

(S-Mebpop)(R,R-dpen)RuCl₂

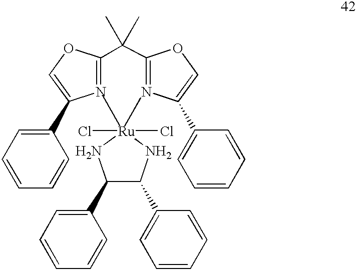

¹H-NMR (300 MHz)(CDCl₃)(−65° C.) δ1.83 (s br, 6H),3.15-3.30 (m,2H),

3.34-3.55 (m,2H),4.15-4.3 (m,2H),4.8-4.95 (m,2H),5.55-5.72 (m,2H),

6.30-6.55 (m,4H),6.3-7.3 (m,6H),7.3 (s br,10H) ppm; ESI-MS: m/z 718;

Purity according to ESI-MS ca. 98%

(S-i-Prbpop)(R,R-dpen)RuCl₂

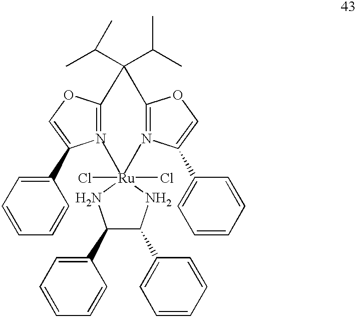

¹H-NMR (300 MHz)(CDCl₃)(−55° C.)δ1.0-1.35 (m,6H),2.55-2.70 (m,2H),

3.1 (s br,4H),3.25-3.45 (m,2H),4.0-4.2 (m,2H),4.8-5.0 (m,2H), 5.75-5.95 (m,2H), 6.3-6.5 (m,4H),6.9-7.1 (m,6H),7.2-7.4 (m,6H),7.45-7.6 (m,4 H) ppm; ESI-MS: m/z 774

Purity according to ESI-MS ca 98%

(S-Mebpo-tert.Bu)(R,R-DPEN) RuCl₂

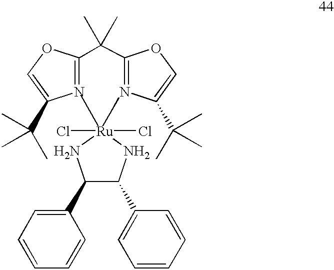

ESI-MS: m/z 678; Purity according to ESI-MS ca. 80%

General Procedure for Hydrogenation of Acetophenone (Results in Table 2)

The reagents and solvents in the amounts indicated in Table 2 were dried and then degassed by several freeze-pump-thaw cycles. Hydrogenations of acetophenone by the Ru-oxazoline-DPEN complexes were carried out under standardized conditions of 0.0055 mmol catalyst (˜4 mg, 1 eq.), 11.1 mmol acetophenone (1.33 g, 2000 eq.), and 0.11 mmol alkali metal t-butoxide (20 eq.) in 3 ml isopropanol at 50° C. under 5 bar H₂ with vigorous stirring. Hydrogen consumption was monitored under isobaric conditions and displayed a short induction time, followed by a broad plateau, and then, typically after 24-60 hours, by a rapid falloff as acetophenone was completely consumed,. Crude product analysis was performed by thin-layer chromatography on Merck Kieselgel plates with 1:5 t-butylmethyl ether/hexane. If hydrogenation is carried out until no further hydrogen consumption is noted, 1-phenylethanol can be isolated in ˜95% yield based on acetophenone. Optical purity of the 1-phenylethanol (enantiomeric excess, e.e) was measured by capillary gas chromatography with a chiral column (CE Instruments GC8000 TOP, 30 m×0.25 mm Supelco beta-DEX 120 column, injector at 200° C., FID detector at 250° C.).

The results of Table 2 demonstrate that catalytic hydrogenation using H₂ as the hydride source can be achieved using Ru-based catalysts with amine ligands and does not require the presence of phosphine ligands. The presence of phenyl or other aryl substituents on the oxazoline ligand of the Ru catalyst enhances reactivity significantly as demonstrated by a comparison of the results in entries 1 and 9 of Table 2. It is believed that an aryl-aryl cation binding site is important in achieving high catalyst activity.. The results indicate that there is cooperative action reaction between chiral ligands for achieving high enantiomer excess in the as shown by a comparison of the results of entries 3 and 5 of Table 2. The product in entry 3 in which the S-phenyloxazoline ligand is paired with the R,R-diphenylethylenediamine ligand exhibits significantly higher enantiomeric excess compared to the product formed (entry 5) using the catalyst carrying the S-phenyloxazoline ligand and the S,S- diphenylethylenediamine ligands.

TABLE 2
Relative rates of H₂ (bar h⁻¹) consumption in the catalytic hydrogenation of
acetophenone with various Ru-oxazoline-dpen complexes*

| | | | S/C/Base | Rate | |
|---|---|---|---|---|---|
| Entry | Ru-oxazoline-dpen catalyst | Base | (mole ratio) | (actvity) | e.e (%) |
| 1 | (S-Mebpop)(R,R-dpen)RuCl₂ | CsOtBu | 2000/1/20 | 0.50 | 50 |
| 2 | (S-Mebpop)(R,R-dpen)RuCl₂ | RbOtBu | 2000/1/20 | 0.50 | |
| 3 | (S-Mebpop)(R,R-dpen)RuCl₂ | CsOtBu | 2000/1/20 | 0.90 | 43.3 |
| 4# | (S-Mebpop)(R,R-dpen)RuCl₂ | CsOtBu | 2000/1/20 | 0.20 | 63.3 |
| 5 | (S-Mebpop)(S,S-dpen)RuCl₂ | CsOtBu | 2000/1/20 | 0.90 | 8.8 |
| 6 | (S-i-Prbpop)(R,R-dpen)RuCl₂ | KOtBu | 2000/1/40 | 3.0 | 31.3 |
| 7# | (S-i-Prbpop)(R,R-dpen)RuCl₂ | KOtBu | 2000/1/40 | 0.50 | 66 |
| 8 | (S-i-Prbpop)(R,R-dpen)RuCl₂ | RbOtBu | 2000/1/40 | 0.75 | |
| 9 | (S-Mebpo-t-Bu)(R,R-dpen)RuCl₂ | CsOtBu | 2000/1/20 | 0.06 | |

*Reactions were run at 50° C., unless otherwise noted.
#Reactions run at 30° C.

Those of ordinary skill in the art will appreciate that materials and methods other than those specifically disclosed herein can be employed in the practice of the invention as broadly described herein. Materials including ligands, bases, salts, solvents, reaction conditions, and procedures that are appreciated in the art to be functionally equivalent to those specifically disclosed herein are intended to be encompassed by this invention. All references cited herein are incorporated by reference herein to the extent that they are not inconsistent with the disclosure herein.

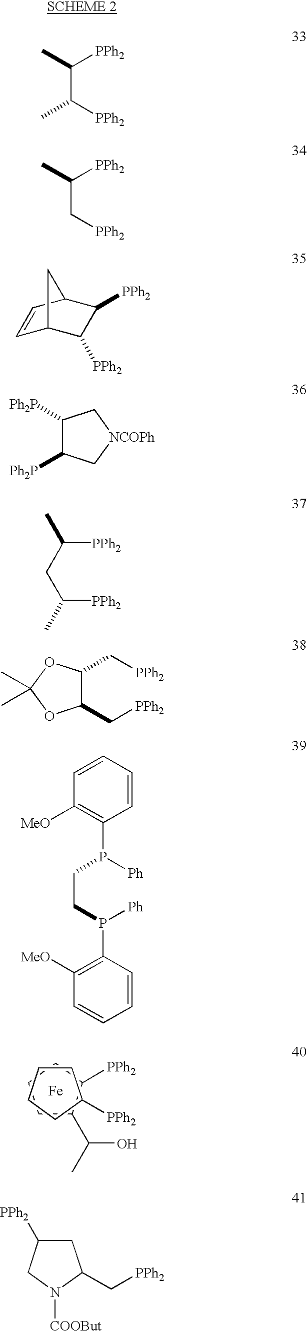

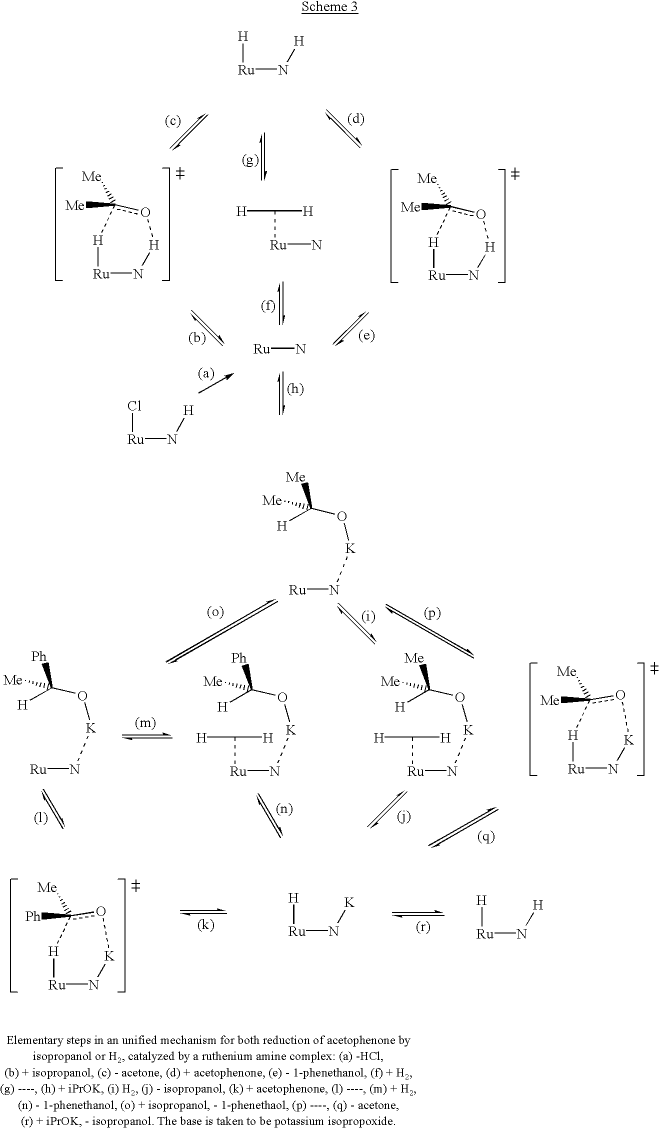

We claim:

1. A method for catalytic hydrogenation which comprises the steps of:
providing a hydrogen-cleaving Ru(II) catalyst by combining a Ru(II) complex of formula:
Ln—Ru—[—X—L″p]n′
wherein:
X is a heteroatom;
L″ is an optionally substituted hydrocarbyl group;
p is an integer dependent upon the valency of X and L″;
L, independent of other L in the complex, is a ligand selected from a halogen, a solvent molecule, a ligand containing one or more oxygen, nitrogen or phosphorous chelating atoms, an arene and dihydrogen; and
n and n′ are integers with n′ ranging from 1 to 6 and n ranging from 0 to 5 dependent upon the valency of L ligands and the value of n′;
with at least one equivalent with respect to the Ru complex of a base and a Lewis acid present in excess over the base; and
contacting a compound to be hydrogenated with the catalyst in the presence of a hydride source.

2. The method of claim 1 wherein the hydride source is hydrogen.

3. The method of claim 1 wherein the base is present in an amount ranging from at least 1 equivalent to about 100 equivalents with respect to the Ru complex.

4. The method of claim 1 wherein the base is present in an amount ranging from at least 10 equivalents to about 100 equivalents with respect to the Ru complex.

5. The method of claim 1 wherein the base is present in an amount ranging from about 10 equivalent to about 50 equivalents with respect to the Ru complex.

6. The method of claim 1 wherein the base is hydroxide, an alkoxide or a hydride base.

7. The method of claim 1 wherein the base is an alkoxide.

8. The method of claim 1 wherein the Lewis acid is an alkali or alkaline earth metal cation.

9. The method of claim 1 wherein the Lewis acid comprises boron, aluminum or tin.

10. The method of claim 9 wherein the Lewis acid is a boronate or an aluminate.

11. The method of claim 1 wherein the Lewis acid comprises a transition metal.

12. The method of claim 1 wherein the Ru(II) complex comprises one or more phosphine ligands.

13. The method of claim 1 wherein the RU(II) complex comprises one or more amine ligands.

14. The method of claim 1 wherein the Ru(II) complex comprises one or more ether ligands.

15. The method of claim 1 wherein the Ru(II) complex comprises an oxazo line ligand.

16. The method of claim 1 wherein the Ru(II) complex comprises an oxazoline ligand and a diamine ligand.

17. The method of claim 1 wherein the Ru(II) complex comprises a π-bonded arene.

18. The method of claim 1 wherein X is nitrogen or oxygen.

19. The method of claim 1 wherein L″ comprises boron, aluminum or tin.

20. The method of claim 1 wherein one or two of L are halogens.

21. The method of claim 1 wherein one or more of L or L″ comprise one or more aryl groups.

22. The method of claim 1 wherein one or more of L and one or more of L″ is a chiral ligand.

23. The method of claim 1 wherein at least one of L is an arene.

24. The method of claim 1 wherein one of L is dihydrogen.

25. The method of claim 1 wherein at least one of L is selected from the group of boron-containing ligands having formulas 11-18 as illustrated in the specification herein wherein R is a hydrocarbyl and the lines linking P and O atoms or two P atoms are hydrocarbyl groups and Ar, if present, is an optionally substituted aryl group.

26. The method of claim 1 wherein at least one of L is selected from the group of phosphorous-containing ligands or enantiomers thereof having formulas 33-41

27. The method of claim 1 wherein the Ru(II) complex is: 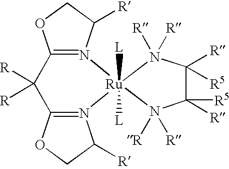
wherein R, independent of other R in the complex, can be hydrogen, an optionally substituted alkyl group or an optionally substituted aryl group; R′, independent of other R′ in the complex, can be an optionally substituted hydrocarbyl group, R″, independent of other R″ in the complex, can be a hydrogen or an optionally substituted hydrocarbyl group; and R⁵, independent of other R⁵ in the complex, can be an optionally substituted hydrocarbyl group wherein any two or more of R, R′, R″ or R⁵ can be linked to form a ring.

28. The method of claim 26 wherein the Ru(II) complexes is: 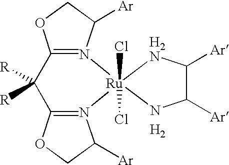
where Ar and Ar′, independently, are optionally substituted aryl groups which can contain one or more aromatic rings, heteroaromatic rings, fused rings or combinations thereof.

29. A method for catalytic hydrogenation which comprises the steps of:
(a) providing a Ru(II) catalyst of formula: 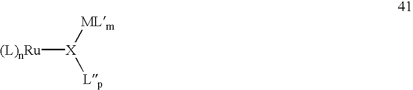
or of formula: 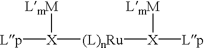
wherein:
X is a heteroatom; L″, independent of other L″, is an optionally substituted hydrocarbyl group; M, independent of other M, is an electrophilic metal atom or cation; L′, independent of other L′ is a ligand selected from a solvent molecule, a halogen, an optionally substituted alkoxide, an optionally substituted aryloxide, an anionic ligand containing one or more halogen, one or more oxygen, or one or more nitrogen atoms or a ligand containing one or more oxygen, one or more nitrogen, one or more phosphorous atoms or a combination of oxygen, nitrogen or phosphorous atoms; Ru is a ruthenium atom in the +2 oxidation state; L is a ligand selected from a halogen, a solvent molecule, dihydrogen, a ligand containing one or more oxygen, nitrogen, and/or phosphorus chelating atoms, a π-bonded optionally substituted arene, a ligand containing one or more oxygen, one or more nitrogen or one or more phosphorous atoms in combination, and an X-L″ₚ group, any two or more of L, L′ or L″ can be linked to form a ring and any of L, L′ or L″ can be chiral; and the integers n, m and p are the number of ligands L, L′ and L″, respectively, and range generally from 0 to 6 dependent upon the valency of the ligands, X, and M; and
b) contacting a compound to be hydrogenated with the catalyst in the presence of a hydride source to thereby hydrogenate the compound.

30. The method of claim 29 wherein M is an alkali or alkaline earth metal cation, a second transition metal, boron, aluminum, or tin.

31. The method of claim 29 wherein the catalyst comprises any one of the complexes of formulas 11-16,19A or B as defined in the specification.

32. The method of claim 29 wherein at least one L, alone or in combination with at least one other L, one L′ or one L″ forms a ligand selected from the group represented by the formulas 17,18, and 20-41 as defined in the specification.

33. The method of claim 29 wherein L, L′ and L″ do not contain phosphorous atoms.

34. The method of claim 29 wherein one or more of L are dihydrogen.

35. The method of claim 29 wherein X is a nitrogen or an oxygen.

36. The method of claim 29 wherein one of L is a π-bonded optionally substituted arene.

37. A hydrogenation catalyst) catalyst of formula: 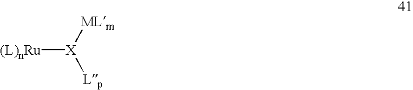
or of formula: 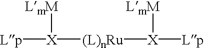
wherein:
X is a heteroatom; L″, independent of other L″, is an optionally substituted hydrocarbyl group; M, independent of other M, is an electrophilic metal atom or cation; L′, independent of other L′ is a ligand selected from a solvent molecule, a halogen, an optionally substituted alkoxide, an optionally substituted aryloxide, an anionic ligand containing one or more halogen, one or more oxygen, or one or more nitrogen atoms or a ligand containing one or more oxygen, one or more nitrogen, one or more phosphorous atoms or a combination of oxygen, nitrogen or phosphorous atoms; Ru is a ruthenium atom in the +2 oxidation state; L is a ligand selected from a halogen, a solvent molecule, dihydrogen, a ligand containing one or more oxygen, nitrogen, and/or phosphorus chelating atoms, a n-bonded optionally substituted arene, a ligand containing one or more oxygen, one or more nitrogen or one or more phosphorous atoms in combination, and an X-L″_{P} group, any two or more of L, L′ or L″ can be linked to form a ring and any of L, L′ or L″ can be chiral; and the integers n, m and p are the number of ligands L, L′ and L″, respectively, and range generally from 0 to 6 dependent upon the valency of the ligands, X, and M; or of formula:

38. The catalyst of claim 37 wherein M is an alkali or alkaline earth metal cation, a transition metal, boron, aluminum, or tin.

39. The catalyst of claim 37 which is has any of formulas 11-16 as defined in the specification.

40. The catalyst of claim 37 wherein at least one L, alone or in combination with one or more other L, one or more other L′ or one or more other L″ is a ligand selected from the group represented by the formulas 17,18 and 20-41 as defined in the specification.

41. The catalyst of claim 37 wherein one or more L are dihydrogen.

42. The catalyst of claim 37 wherein L, L′ and L″ do not contain phosphorous atoms.

43. The catalyst of claim 37 wherein one of L is a π-bonded optionally substituted arene.

44. A method for catalytic hydrogenation of a compound which comprises the steps of:
(a) combining a first component containing the Ru (II) complex structure: 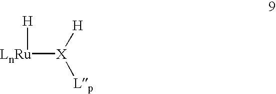
wherein:
X is a hetero atom; L″, independent of other L″, is an optionally substituted hydrocarbyl group; Ru is a ruthenium atom in the +2 oxidation state; L is a ligand selected from a halogen, a solvent molecule, dihydrogen, a ligand containing one or more oxygen, nitrogen, and/or phosphorus chelating atoms, a π-bonded optionally substituted arene, a ligand containing one or more oxygen, one or more nitrogen or one or more phosphorous atoms in combination, and an X-L″ₚ group, any two or more of L, or L″ can be linked to form a ring and any of L, or L″ can be chiral; and the integers n, and p are the number of ligands L, and L″, respectively, and range generally from 0 to 6 dependent upon the valency of the ligands, and X,
with more than one equivalent with respect to the Ru(II) complex of a second component that is a Lewis acid; and
(b) contacting a compound to be hydrogenated with the combination in the presence of a hydride source to thereby hydrogenate the compound.

45. The method of claim 44 wherein the second component is either (1) a salt in which the cation is an electrophilic metal cation and the anion is a weakly or noncoordinating anion, or (2) a neutral Lewis-acidic compound.

46. The method of claim 44 wherein at least one L, alone or in combination with at least one other L or at least one L″ is a ligand selected from the group represented by the formulas 17,18,20-41 as defined in the specification.

47. The method of claim 44 wherein more than 1 to about 100 equivalents of the second component are combined with the first component.

48. A method for hydrogenating a compound which comprises the steps of:
(a) combining a first component containing the Ru(II) complex structure: 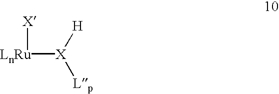
wherein:
X is a heteroatom; L″, independent of other L″, is an optionally substituted hydrocarbyl group; X′ is a good leaving group; Ru is a ruthenium atom in the +2 oxidation state; L is a ligand selected from a halogen, a solvent molecule, dihydrogen, a ligand containing one or more oxygen, nitrogen, and/or phosphorus chelating atoms, a π-bonded optionally substituted arene, a ligand containing one or more oxygen, one or more nitrogen or one or more phosphorous atoms in combination, and an X-L″ₚ group, any two or more of L, or L″ can be linked to form a ring and any of L, or L″ can be chiral; and the integers n, and p are the number of ligands L, and L″, respectively, and range generally from 0 to 6 dependent upon the valency of the ligands, X′ and X,
with one or more equivalents with respect to the Ru(II) complex of a second component which is a base and with more than one equivalent with respect to the base of a third component that is a Lewis acid and contacting the combination with a hydride source and the compound to be hydrogenated to thereby hydrogenate the compound.

49. The method of claim 48 wherein the third component is either (1) a salt in which the cation is an electrophilic metal cation and the anion is a weakly coordinating or noncoordinating anion, or (2) a neutral Lewis-acidic compound.

50. The method of claim 48 wherein the base is selected from a hydroxide, alkoxide, or hydride base.

51. The method of claim 48 wherein the base is combined at a level of 10 to 100 equivalents with respect to the Ru(II) complex.

52. The method of claim 48 wherein the third component is combined with the first and third components at a level of more than one to about five equivalents with respect to the base.

53. A catalytic formulation which is prepared by combining a Ru(II) complex of formula:
Ln—Ru—[—X—L″p]n′
wherein:
X is a heteroatom;
L″ is an optionally substituted hydrocarbyl group;
p is an integer dependent upon the valency of X and L″;
L, independent of other L in the complex, is a ligand selected from a halogen, a solvent molecule, a ligand containing one or more oxygen, nitrogen or phosphorous chelating atoms, an arene and dihydrogen; and
n and n′ are integers with n′ ranging from 1 to 6 and n ranging from 0 to 5 dependent upon the valency of L ligands and the value of n′;
with at least one equivalent with respect to the Ru complex of a base and more than one equivalent with respect to the base of a Lewis acid.

54. The catalytic formulation of claim 53 which is prepared in an alcohol solvent.

55. The catalytic formulation of claim 53 which is prepared in the presence of a hydride source.

56. The catalytic formulation of claim 53 which is prepared in the presence of hydrogen.

57. The catalytic formulation of claim 53.wherein the base is present in an amount ranging from at least about 10 equivalents to about 100 equivalents with respect to the Ru complex.

58. The catalytic formulation of claim 53 wherein the base is hydroxide, an alkoxide or a hydride base.

59. The catalytic formulation of claim 53 wherein the Lewis acid is an alkali or alkaline earth metal cation.

60. The catalytic formulation of claim 53 wherein the Lewis acid comprises boron, aluminum or tin.

61. The catalytic formulation of claim 53 wherein the Lewis acid is a boronate or an aluminate.

62. The catalytic formulation of claim 53 wherein the Lewis acid comprises a transition metal.

63. The catalytic formulation of claim 53 wherein the Ru(II) complex comprises an oxazoline ligand.

64. The catalytic formulation of claim 53 wherein the Ru(II) complex comprises an oxazoline ligand and a diamine ligand.

65. The catalytic formulation of claim 53 wherein the Ru(II) complex comprises a π-bonded arene.

66. The catalytic formulation of claim 53 wherein one or more of L or L″ comprise one or more aryl groups.

67. The catalytic formulation of claim 53 wherein one or more of L and one or more of L″ are chiral ligands.

68. The catalytic formulation of claim 53 wherein L does not contain phosphorous.

69. The catalytic formulation of claim 53 wherein the Ru(II) complex is: 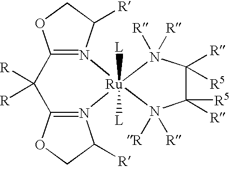
wherein R, independent of other R in the complex, can be hydrogen, an alkyl group or an aryl group; R′, independent of other R′ in the complex, can be a hydrocarbyl group, R″, independent of other R″ in the complex, can be a hydrogen or a hydrocarbyl group; and R⁵, independent of other R⁵ in the complex, can be hydrocarbyl groups wherein any two or more of R″ or R⁵ can be linked to form a ring.

70. The catalyti c formulation of claim 53 wherein the RU(II) complexes is: 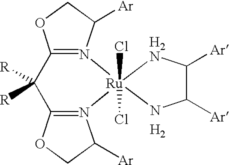
where Ar and Ar′, independently, are aryl groups which can contain one or more aromatic ring which can include heteroaromatic and/or fused rings, any of which may be substituted with one or more halogens, one or more alkyl groups, one or more alkoxy groups or one or more halogenated alkyl groups.

71. A catalytic formulation which is prepared by combining a first component containing the Ru(II) complex structure: 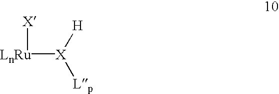
wherein:
X is a heteroatom; L″, independent of other L″, is an optionally substituted hydrocarbyl group; X′ is a good leaving group; Ru is a ruthenium atom in the +2 oxidation state; L is a ligand selected from a halogen, a solvent molecule, dihydrogen, a ligand containing one or more oxygen, nitrogen, and/or phosphorus chelating atoms, a π-bonded optionally substituted arene, a ligand containing one or more oxygen, one or more nitrogen or one or more phosphorous atoms in combination, and an X-L″ₚ group, any two or more of L, or L″ can be linked to form a ring and any of L, or L″ can be chiral; and the integers n, and p are the number of ligands L, and L″, respectively, and range generally from 0 to 6 dependent upon the valency of the ligands, X′ and X,
with one or more equivalents with respect to the Ru(II) complex of a second component which is a base and with more than one equivalent with respect to the base of a third component that is a Lewis acid.

72. The catalytic formulation of claim 71 which is prepared in an alcohol solvent.

73. The catalytic formulation of claim 71 which is prepared in the presence of a hydride source.

74. The catalytic formulation of claim 71 which is prepared in the presence of hydrogen.

75. A catalytic formulation prepared by combining
(a) a first component containing A method for catalytic hydrogenation of a compound which comprises the steps of:
(a) combining a first component containing the Ru (II) complex structure: 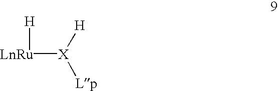
wherein:
X is a heteroatom; L″, independent of other L″, is an optionally substituted hydrocarbyl group; Ru is a ruthenium atom in the +2 oxidation state; L is a ligand selected from a halogen, a solvent molecule, dihydrogen, a ligand containing one or more oxygen, nitrogen, and/or phosphorus chelating atoms, a π-bonded optionally substituted arene, a ligand containing one or more oxygen, one or more nitrogen or one or more phosphorous atoms in combination, and an X-L″_{P} group, any two or more of L, or L″ can be linked to form a ring and any of L, or L″ can be chiral; and the integers n, and p are the number of ligands L, and L″, respectively, and range generally from 0 to 6 dependent upon the valency of the ligands, and X,
with more than one equivalent with respect to the Ru(II) complex of a second component that is a Lewis acid.

76. The catalytic formulation of claim 75 which is prepared in an alcohol solvent.

77. The catalytic formulation of claim 75 which is prepared in the presence of a hydride source.

78. The catalytic formulation of claim 75 which is prepared in the presence of hydrogen.

79. A Ru(II) complex having the formula: 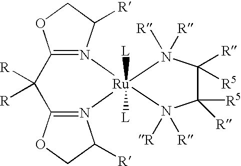
wherein R, independent of other R in the complex, can be hydrogen, an optionally substituted alkyl group or an optionally substituted aryl group; R′, independent of other R″ in the complex, can be an optionally substituted hydrocarbyl group, R″, independent of other R″ in the complex, can be a hydrogen or an optionally substituted hydrocarbyl group; and R⁵, independent of other R⁵ in the complex , can be an optionally substituted hydrocarbyl group wherein any two or more of R, R′, R″ or R⁵ can be linked to form a ring.

80. The method of claim 79 wherein the Ru(II) complexes is: 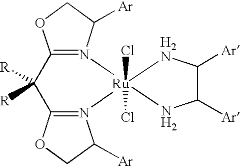
where Ar and Ar′, independently, are optionally substituted aryl groups which can contain one or more aromatic ring, heteroaromatic rings, fused rings or combinations thereof.
